(19) United States
(12) Patent Application Publication
(10) Pub. No.: US 2013/0190698 A1
(43) Pub. Date: Jul. 25, 2013

(54) Device for Use in Clinical Irrigation

(71) Applicant: Desmond Carson, Richmnd, CA (US)

(72) Inventor: Desmond Carson, Richmnd, CA (US)

(21) Appl. No.: 13/357,621

(22) Filed: Jan. 25, 2012

(51) Int. Cl. A61M 3/02

(52) U.S. Cl. 604/257

(57) ABSTRACT

Methods and devices for use in clinical irrigation are provided. The present invention further provides compositions and devices for use in irrigation and cleaning of wounds.

### FIELD OF THE INVENTION

The present invention relates to devices and method used in clinical irrigation.

### BACKGROUND OF THE INVENTION

The primary goal in wound management is to achieve rapid healing with optimal functional and aesthetic results. This goal is best accomplished by preventing infection and further trauma, and providing an environment that optimizes healing. Conventional treatment for wounds has been thorough cleansing with clean water or normal saline.

Certain types of wounds are best treated by high pressure irrigation.

For highly contaminated wounds, continuous high-pressure irrigation is typically used. Current methods of high pressure irrigation may use a 10-60 ml syringe and splatter shield. Other current methods include the use of a 30-60 ml syringe and a 19-gauge needle and splash shield.

Pressurized irrigation of wounds offers myriad documented benefits in healing.

It would be desirable to provide an irrigation device and procedure that may effectively continually irrigate a wound with minimal effort on the part of the clinician. It also would be desirable for that device to be easily connected to a container of medical grade saline, so that fresh sterile saline supplies may be replaced with minimal effort or time on the part of the clinician or clinical staff. It is among the overall objects of the invention to provide such a device and methods.

### SUMMARY OF THE INVENTION

In certain embodiments, a device for the irrigation of wounds is provided, wherein the device comprises: a port (1) for the attachment an oxygen supply (2), an area (3) that sealably attaches to a supply of sterile saline (4), and a tube portion (5), wherein sterile saline may be sent through the tube portion (5) and out of a nozzle (6), wherein the nozzle (6) may be directed to irrigate a wound.

In certain embodiments, the invention further comprises a device wherein the tube portion (5) and nozzle (6) permit sterile saline to be moved out of the nozzle at a pressure of 5 to 8 psi.

In certain embodiments, the invention further comprises a device wherein the tube portion (5) and nozzle (6) permit sterile saline to be moved out of the nozzle at a pressure of 7 to 8 psi.

In certain embodiments, the device further comprises a means (7) to modulate the pressure of sterile saline flowing through the nozzle (6).

In certain embodiments, the invention further comprises a device wherein the area (3) that sealably attaches to a supply of sterile saline is an aperture with internal threads, wherein the internal threads are capable of screwing onto a bottle of commercially available medical grade saline.

In certain embodiments, the device which further comprises a mechanism (8) which allows an operator to easily turn the flow of sterile saline on and off. In certain embodiments, the mechanism (8) comprises a trigger.

In certain embodiments, the invention further comprises a device wherein the port (1) attaches to a pressurized oxygen supply.

In certain embodiments, a device for the irrigation of wounds is provided, wherein the device comprises: a port (1) for the attachment a pressurized oxygen supply (2); an area (3) that sealably attaches to a supply of sterile saline (4), the area (3) comprising an aperture with internal threads, wherein the internal threads are capable of screwing onto a bottle of commercially available medical grade saline; and a tube portion (5), wherein sterile saline may be sent through the tube portion (5) and out of a nozzle (6) at a pressure of 7 to 8 psi, wherein the nozzle (6) may be directed to irrigate a wound.

### BRIEF DESCRIPTION OF THE DRAWINGS

The foregoing and other objects and advantages of the invention will be appreciated more fully from the following further description thereof, with reference to the accompanying drawings wherein:

FIG. 1 shows a minimal iteration of a certain embodiment of the present invention.

FIG. 2 shows a certain embodiment of the invention further comprising a trigger mechanism (8) which allows an operator to turn the flow of sterile saline on and off, and further comprising a means (7) to modulate the pressure of sterile saline flowing through the nozzle (6).

### DETAILED DESCRIPTION OF THE INVENTION

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “piece” or “component” encompass both pieces and components comprising one unit and pieces and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present invention comprises a port (1) for the attachment of an oxygen supply (2). It is envisioned that the pressure generated by the oxygen supply pushes the sterile saline out through the tube portion (5) and out of the nozzle (6). In certain embodiments, the port (1) attaches to a pressurized oxygen supply hose. In certain embodiments, the port (1) is adaptable in a manner such that it may be attached to a variety of diameters of oxygen supply hoses. The port may be any opening, whether adjustable or not through which oxygen, air, or any other pressurized gas may flow from a pressurized oxygen supply into the supply of sterile saline. In certain embodiments, the port sealably attaches to the oxygen supply such that oxygen from the oxygen supply may flow only into the supply of sterile saline.

In certain embodiments, the port (1) comprises a flexible tubing, the distal end of which may attach directly onto an oxygen supply.

The present invention comprises an area that sealably attaches to a supply of sterile saline. Several configurations are envisioned for the area that sealably attaches to a supply of sterile saline. In certain embodiments, the area that sealably attaches to a supply of sterile saline attaches to the supply of sterile saline in a manner identical to a cap for the supply of sterile saline. In certain, the supply of sterile saline may be a bottle of commercially available medical grade saline, and the area that sealably attaches to the supply of sterile saline comprising an aperture with internal threads, wherein the internal threads are capable of attaching to a bottle of commercially available medical grade saline. In certain embodiments, the area that sealably attaches to the supply of sterile saline comprises an aperture with a quick locking mechanism.

In certain embodiments, the area that sealably attaches to a supply of sterile saline comprises a quick release mechanism which reduces the time required to replace the supply of sterile saline. It is envisioned that such an area might easily detach from an empty supply of sterile saline and be reattached to a fresh supply of sterile saline quickly.

In certain embodiments, the area that sealably attaches to a supply of sterile saline comprises an area that fits inside the opening of the supply of sterile saline. In certain embodiments, alternative solutions may be substituted for medical grade sterile saline. As an exemplary embodiment, an alternative solution may contain supplements, including, but not limited to, antibiotics or antifungal medication.

The present invention further comprises a tube portion (5), wherein a first end (9) of the tube portion (5) is submerged in the sterile saline, and a second end (10) of the tube portion ends comprises a nozzle (6), and wherein sterile saline may be sent through the tube portion (5) and out of the nozzle (6), wherein the nozzle (6) may be directed to irrigate a wound. In certain embodiments, it may be desirable that the first end (9) is of a sufficient length that it extends to near the bottom the supply of sterile saline in a manner such that the first end (9) remains submerged in sterile saline until the container holding the supply of sterile saline is nearly empty. In certain embodiments, the device of the present invention ejects a saline spray at a pressure of 5 to 8 psi. In certain embodiments, the device of the present invention ejects a saline spray at a pressure of 7 to 8 psi. In certain embodiments, the device of the present invention ejects a saline spray at a pressure of 7 to 7.5 psi.

In certain embodiments, the tube portion (5) further comprises a removable sheath which covers the nozzle (6). In certain embodiments, the sheath may further be removed from the tube portion (5) and replaced after use.

In certain embodiments, the nozzle (6) has a fixed nonadjustable aperture which has been calibrated to eject saline at specified pressure when the present invention is attached to a standard pressurized oxygen supply. In certain embodiments, the tube portion (5) further comprises a removable sheath which covers the nozzle (6), wherein the removable sheath has a fixed nonadjustable aperture which has been calibrated to eject saline at specified pressure when the present invention is attached to a standard pressurized oxygen supply.

In accordance with the invention, the tube portion (5) may be made of a flexible plastic, such as polyvinyl chloride (PVC), polyamide (PA) polymers such as nylon, natural and synthetic latex. Other suitable polymers may be used, without limitation. In certain embodiments, the tube portion (5) may be made of Pyrex glass. In certain embodiments, the tube portion (5) may be made of surgical grade steel. It is within the scope of the invention that individual components of the device may be assembled from separate components. It is within the scope of the invention that any component of the present invention may be made of surgical grade steel. It is further envisioned that the several components may be composed of differing substances, including, but not limited to, polymers, metals, metal alloys, and glass. It is within the scope of the invention that any two or more components of the device, in whole or in part, may be cast as a single piece from polymers. It is within the scope of the invention that any two or more components of the device, in whole or in part, may be cast as a single piece from any material, such as glass or surgical grade steel.

### EXEMPLARY EMBODIMENTS

The following embodiments are for illustrative purposes, and are not meant to limit the present invention in any manner.

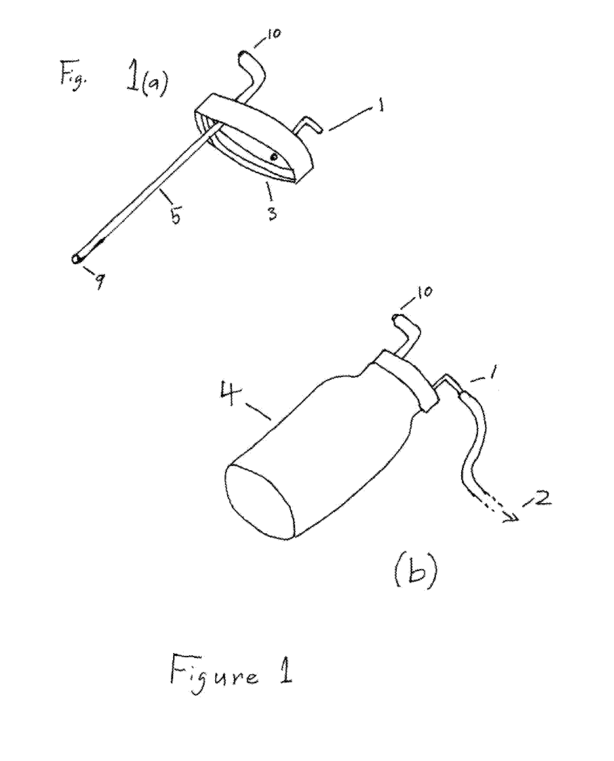

FIG. 1 shows an exemplary embodiment that may, in certain embodiments, be cast from a single polymer. The area (3) that sealably attaches to a supply of sterile saline (4) comprises a circular portion wherein the diameter of the circular portion is identical to the diameter of a cap of a bottle of commercially available sterile saline, and wherein the circular portion comprises internal threads identical in spacing and pitch to the internal threads of the cap of a bottle of commercially available sterile saline.

The exemplary device further comprises a port (1) for the attachment of an oxygen supply. The port (1) may be readily attached to a variety of hose diameter attached to a pressurized oxygen supply. The port is attached to the top of the circular portion. The exemplary device further comprises a tube portion (5), wherein a first end (9) of the tube portion (5) is submerged in the sterile saline, and a second end (10) of the tube portion ends comprises a nozzle (6).

FIG. 1a shows the exemplary embodiment alone, while FIG. 1b shows the exemplary embodiment attached to a an oxygen supply and a supply of sterile saline (4).

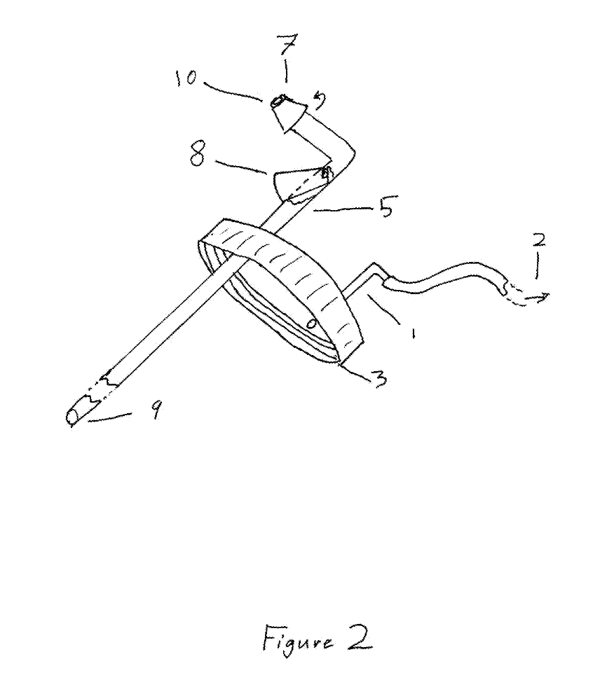

FIG. 2 shows a further exemplary embodiment. The exemplary device of the present invention comprises a trigger mechanism (8) which permits the outflow of sterile saline when the trigger is depressed.

The exemplary device further comprises a port (1) for the attachment of an oxygen supply. The port (1) may be readily attached to a variety of hose diameter attached to a pressurized oxygen supply. The exemplary device further comprises a tube portion (5), wherein a first end (9) of the tube portion (5) is submerged in the sterile saline, and a second end (10) of the tube portion ends comprises a nozzle (6).

The exemplary device further comprises a means (7) for modulating the pressure at which the sterile saline flows out of the nozzle (6). The means comprises an adjustable valve which adjusts the aperture through which oxygen flows, which in turn adjusts the pressure of the out flowing sterile saline.

The system should be readily assembled and provide a continuous flow of sterile saline to the wound as desired by the clinician. It should be understood, however, that the foregoing description of the invention is intended merely to be illustrative thereof and that other embodiments and modifications may be apparent to those skilled in the art without departing from its spirit.

From the foregoing, it should be appreciated that the invention provides an improved irrigation device for use in irrigating wounds. Several further modifications known in the medical arts may further be contemplated within the device of the present invention.

What is claimed is:

1. A device for the irrigation of wounds, wherein the device comprises:
a port (1) for the attachment an oxygen supply (2), an area (3) that sealably attaches to a supply of sterile saline (4), and a tube portion (5), wherein sterile saline may be sent through the tube portion (5) and out of a nozzle (6), wherein the nozzle (6) may be directed to irrigate a wound.

2. A device of claim 1, wherein the tube portion (5) and nozzle (6) permit sterile saline to be moved out of the nozzle at a pressure of 5 to 8 psi.

3. A device of claim 1, wherein the tube portion (5) and nozzle (6) permit sterile saline to be moved out of the nozzle at a pressure of 7 to 8 psi.

4. A device of claim 1, wherein the device further comprises a means (7) to modulate the pressure of sterile saline flowing through the nozzle (6).

5. A device of claim 1, wherein the area (3) that sealably attaches to a supply of sterile saline is an aperture with internal threads, wherein the internal threads are capable of screwing onto a bottle of commercially available medical grade saline.

6. A device of claim 1, which further comprises a mechanism (8) which allows an operator to turn the flow of sterile saline on and off.

7. A device of claim 6, wherein the mechanism is a trigger.

8. A device of claim 1, wherein the port (1) attaches to a pressurized oxygen supply.

9. A device for the irrigation of wounds, wherein the device comprises:
a port (1) for the attachment a pressurized oxygen supply (2);
an area (3) that sealably attaches to a supply of sterile saline (4), the area (3) comprising an aperture with internal threads, wherein the internal threads are capable of screwing onto a bottle of commercially available medical grade saline; and
a tube portion (5), wherein sterile saline may be sent through the tube portion (5) and out of a nozzle (6) at a pressure of 7 to 8 psi, wherein the nozzle (6) may be directed to irrigate a wound.
